(19) United States
(12) Patent Application Publication
(10) Pub. No.: US 2024/0346853 A1
(43) Pub. Date: Oct. 17, 2024

(54) INFORMATION PROCESSING SYSTEM, INFORMATION PROCESSING APPARATUS, INFORMATION PROCESSING METHOD, AND NON-TRANSITORY COMPUTER READABLE MEDIUM STORING PROGRAM

(71) Applicant: NEC Corporation, Minato-ku, Tokyo (JP)

(72) Inventors: Yoshifumi Onishi, Tokyo (JP); Koichi Nihei, Tokyo (JP); Takanori lwai, Tokyo (JP)

(73) Assignee: NEC Corporation, Minato-ku, Tokyo (JP)

(21) Appl. No.: 18/294,589

(22) PCT Filed: Sep. 29, 2021
(86) PCT No.: PCT/JP2021/035831

§ 371 Date: Feb. 2, 2024

(51) Int. Cl. G06V 40/60; H04N 23/69

(52) U.S. Cl. CPC G06V 40/63; H04N 23/69

(57) ABSTRACT

An information processing system (100) according to the present disclosure includes acquisition means (101) for acquiring video data including a subject, calculation means (102) for calculating reliability of biometric information of the subject measured from the video data, and instruction means (103) for outputting, to the subject, instruction information for adjusting a state of the subject based on the calculated reliability. The information processing system (100) may further include shooting means for shooting the subject and thereby generating the video data, and zoom control means for controlling a zoom of the shooting means, in which the instruction means (103) changes the instruction information according to a zoom state of the shooting means.

### TECHNICAL FIELD

The present disclosure relates to an information processing system, an information processing apparatus, an information processing method, and a non-transitory computer readable medium storing a program.

### BACKGROUND ART

Patent Literature 1 discloses an apparatus that performs a remote medical examination, in which a doctor asks a patient about his/her condition or the like through a video call and performs auscultation by using an electronic stethoscope. In this apparatus, the display mode of a display on the doctor's side is switched between an inquiry mode and an auscultation mode based on state information of the electronic stethoscope.

The inquiry mode and the auscultation mode are switched according to whether the electronic stethoscope is in an On-state or an Off-state or whether it is performing a measurement or not. On the display on the doctor's side, the face of the patient or his/her diseased part is displayed in a large size in the inquiry mode, and an image including the whole chest of the patient is displayed in the auscultation mode, thus making it easy to recognize which part of the patient is being auscultated.

### CITATION LIST

### Patent Literature

Patent Literature 1: Japanese Unexamined Patent Application Publication No. 2020-081364

### SUMMARY OF INVENTION

### Technical Problem

In a medical service providing a system by which a doctor makes a diagnosis based on a video image of a patient, when the reliability of biometric information measured from the video image of the patient is low, it may lead to a misdiagnosis. Therefore, there is a concern that the quality of the medical service will deteriorate. In Patent Literature 1, the mode of the display on the doctor's side is merely switched between the inquiry mode and the auscultation mode, and does not take the reliability of measured information into consideration.

An object of the present disclosure is to provide an information processing system, an information processing apparatus, an information processing method, and a non-transitory computer readable medium storing a program capable of taking the reliability of biometric information measured from a video image of a subject into consideration.

### Solution to Problem

An information processing system according to an aspect includes: acquisition means for acquiring video data including a subject; calculation means for calculating reliability of biometric information of the subject measured from the video data; and instruction means for outputting, to the subject, instruction information for adjusting a state of the subject based on the calculated reliability.

An information processing apparatus according to an aspect includes: acquisition means for acquiring video data including a subject; calculation means for calculating reliability of biometric information of the subject measured from the video data; and control means for performing control so as to output, to the subject, instruction information for adjusting a state of the subject based on the calculated reliability.

An information processing method according to an aspect includes: acquiring video data including a subject; calculating reliability of biometric information of the subject measured from the video data; and outputting, to the subject, instruction information for adjusting a state of the subject based on the calculated reliability.

A non-transitory computer readable medium according to an aspect stores a program for causing a computer to perform: a process for acquiring video data including a subject; a process for calculating reliability of biometric information of the subject measured from the video data; and a process for outputting, to the subject, instruction information for adjusting a state of the subject based on the calculated reliability.

### Advantageous Effects of Invention

According to the above-described aspect, it is possible to provide an information processing system, an information processing apparatus, an information processing method, and a non-transitory computer readable medium storing a program capable of taking the reliability of biometric information measured from a video image of a subject into consideration.

### BRIEF DESCRIPTION OF DRAWINGS

FIG. 1 is a block diagram showing a configuration of an information processing system according to an example embodiment;

FIG. 2 shows an example of a configuration of an information processing system including an information processing apparatus according to an example embodiment;

FIG. 3 is a block diagram showing an example of a configuration of a doctor-side terminal shown in FIG. 2;

FIG. 4 is a block diagram showing an example of a configuration of a patient-side terminal shown in FIG. 2;

FIG. 5 is a diagram for explaining an example of contents displayed in the doctor-side terminal;

FIG. 6 is a diagram for explaining an example of contents displayed in the patient-side terminal; and

FIG. 7 is a sequence diagram for explaining an example of a flow of an information processing according to an example embodiment.

### EXAMPLE EMBODIMENT

An example embodiment according to the present disclosure will be described hereinafter with reference to the drawings. Note that the following descriptions and drawings have been omitted and simplified as appropriate for clarifying the explanation. Further, the same reference numerals (or symbols) are assigned to the same elements throughout the drawings, and redundant descriptions thereof are omitted as appropriate. Further, specific numerical values and the like shown below are merely examples for facilitating understanding of the present disclosure, and they are not limited to those shown below.

An example embodiment relates to a medical service providing technology in which video data of a subject is transmitted, through a network, between a facility in which medical services are provided and another facility which is remotely located from the aforementioned facility and in which a subject who receives a medical service is present, and an expert such as a doctor makes a diagnosis based on a video image(s) of the subject.

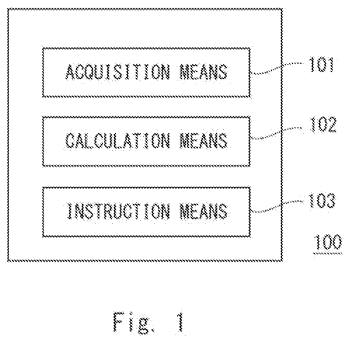

FIG. 1 is a block diagram showing a configuration of an information processing system 100 according to an example embodiment. As shown in FIG. 1, the information processing system 100 includes acquisition means 101, calculation means 102, and instruction means 103. The acquisition means 101 acquires video data including a subject (i.e., containing an image of a subject).

The calculation means 102 calculates the reliability of biometric information of the subject measured from the video data acquired by the acquisition means 101. Based on the reliability calculated by the calculation means 102, the instruction means 103 outputs, for the subject, instruction information for adjusting the state of the subject.

As described above, by outputting instruction information for adjusting the state of the subject while taking the reliability of biometric information measured from the video image of the subject into consideration, the subject himself/herself can adjust his/her state by referring to the instruction information. By measuring biometric information from the video image of the subject in the state where the state of the subject has been adjusted as described above, it is possible to improve the reliability of the biometric information of the subject and thereby to improve the medical service.

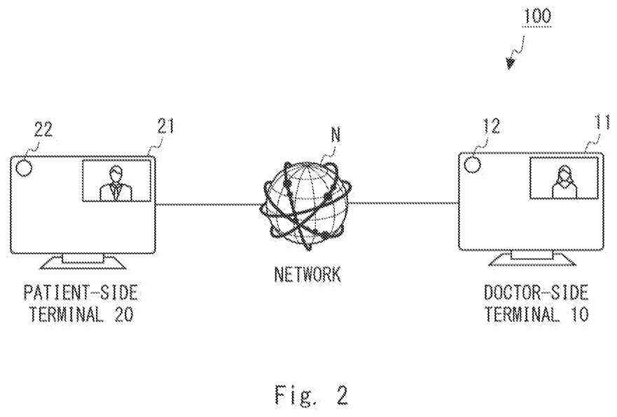

Next, a specific example of the information processing system 100 according to an example embodiment will be described with reference to FIGS. 2 to 4. FIG. 2 shows an example of a configuration of the information processing system 100 including an information processing apparatus 1 according to the example embodiment. As shown in FIG. 2, the information processing system 100 includes a doctor-side terminal 10 and a patient-side terminal 20.

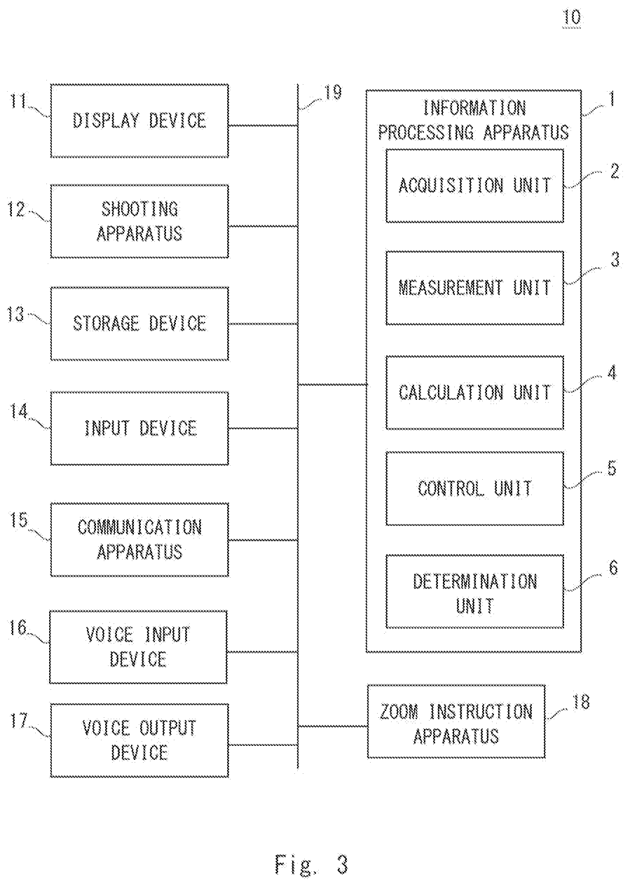

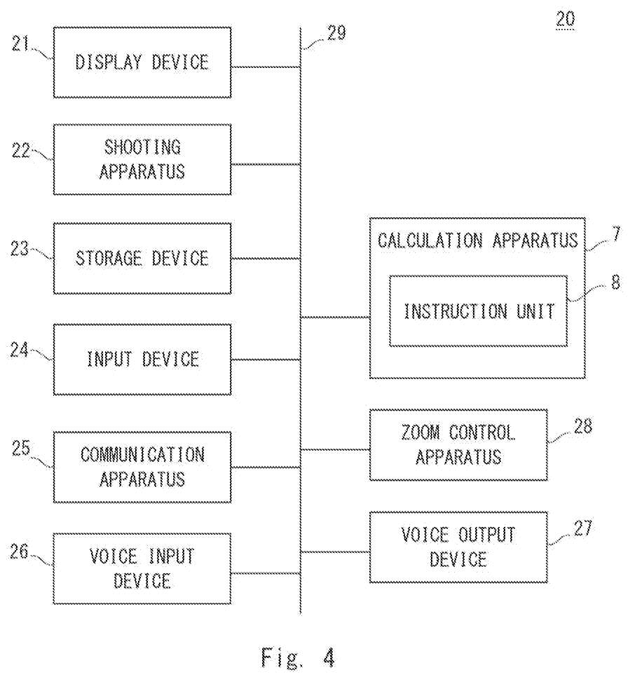

FIG. 3 is a block diagram showing an example of a configuration of the doctor-side terminal 10 shown in FIG. 2. FIG. 3 shows logical functional blocks implemented inside the information processing apparatus 1 as well as a hardware configuration of the doctor-side terminal 10. FIG. 4 is a block diagram showing an example of a configuration of the patient-side terminal 20 shown in FIG. 2. FIG. 4 shows logical functional blocks implemented inside an arithmetic apparatus 7 as well as a hardware configuration of the patient-side terminal 20.

The information processing system 100 supports remote medical care (online medical care or at-home medical care) in which a subject who receives a medical service receives various services from a medical service provider while staying in his/her house. Note that examples of medical service providers include a doctor, a nurse, a physical therapist, and a caregiver. Further, examples of remote medical care (or tele-medical care) include, in addition to medical practices in the narrow sense, such as a medical treatment and procedure performed by a doctor or the like, medical practices in the broad sense, such as a rehabilitation therapy performed by a physical therapist, health management and medicine-taking management performed by a caregiver. A medical service provider is referred to as a “doctor” to simplify the explanation.

Examples of subjects who receive medical services include a patient, a person who undergoes a medical examination (a health examinee), and a person who receives care (a care receiver). A subject who receives a medical service is referred to as a “patient” to simplify the explanation. Further, examples of facilities that provide medical services include a hospital, a clinic, and a nursing home. A facility that provides medical services is referred to as a “hospital” to simplify the explanation.

The doctor-side terminal 10 and the patient-side terminal 20 are connected to each other through a network N, and perform two-way communication in real time. Note that the network N is a communication network such as the Internet or a private line. In the case of the Internet, a closed network is preferably formed by a VPN (Virtual Private Network) or the like.

The doctor-side terminal 10 is installed in a hospital, and the patient-side terminal 20 is installed in a house of a patient remotely located from the hospital. Each of the doctor-side terminal 10 and the patient-side terminal 20 may be a personal computer, a server machine, and a tablet-type terminal and a smartphone capable of inputting/outputting data or the like through a touch panel, or the like. Each of the doctor-side terminal 10 and the patient-side terminal 20 may be a dedicated apparatus, or may be implemented by installing a dedicated application on a general-purpose information terminal. The doctor present in the hospital can, by using the doctor-side terminal 10, communicate with the patient who is in a place remotely located from the hospital and uses the patient-side terminal 20 in real time.

In such remote medical care using the information processing system 100, the doctor makes a diagnosis based on video images transmitted from the patient's home. The doctor-side terminal 10 can acquire biometric information of the patient from a video image(s) obtained by shooting (e.g., capturing a video image of) the patient. Biometric information is information about the living body of a person that can be measured (i.e., obtained) from a video image(s) of the person, and examples thereof include a heart rate, a blood pressure, and oxygen saturation.

The doctor-side terminal 10 can obtain such biometric information based on, for example, information indicating a change in the state of the body surface of the patient shown in a video image of the body surface. An example of information indicating a change in the state of a body surface is a change in the color of the body surface that occurs because of the flow of the blood in the patient. Each frame of video data obtained by shooting a patient includes (i.e., shows) a change in the color of the body surface of the patient. In the doctor-side terminal 10, the doctor or the like can measure a heart rate based on the changes in the color of the body surface of the patient.

Percutaneous arterial oxygen saturation (SpO₂) is a percentage representing how much oxygen is bound with hemoglobin in the blood. The intensity of light that is reflected from the body surface when light is applied to a body surface such as a face changes over time due to changes in the amount of hemoglobin in blood vessels. The doctor-side terminal 10 can estimate SpO₂ by analyzing a video image(s).

If the patient moves relative to the shooting apparatus (e.g., the video camera), e.g., if the patient swiftly moves his/her face, during the measurement of such biometric information, the accuracy of the biometric information measured from video data obtained by shooting the patient deteriorates. In an example embodiment, information necessary for a patient is provided to the patient while taking the reliability of biometric information of the patient into consideration.

As shown in FIG. 3, the doctor-side terminal 10 includes an information processing apparatus 1, a display device 11, a shooting apparatus 12, a storage device 13, an input device 14, a communication apparatus 15, a voice input device 16, a voice output device 17, and a zoom instruction apparatus 18, all of which are connected to each other through a bus 19. The information processing apparatus 1 is an arithmetic processing apparatus such as a CPU (Central Processing Unit) or a GPU (Graphics Processing Unit).

The information processing apparatus 1 includes, as logical functional blocks, an acquisition unit 2, a measurement unit 3, a calculation unit 4, a control unit 5, and a determination unit 6. The acquisition unit 2, the calculation unit 4, and the control unit 5 correspond to acquisition means, calculation means, and control means, respectively, specified in the claims.

The acquisition unit 2 acquires video data including a patient (i.e., containing an image of a patient). The video data including the patient is transmitted, for example, from the patient-side terminal 20 through a network. The video data including the patient may be one that is stored in a database (not shown) connected to the network. In this case, the acquisition unit 2 can acquire the video data by accessing the database.

The measurement unit 3 measures biometric information of the patient from the video data including the patient. In the following description, an example in which a heart rate is measured as biometric information will be described. The measurement unit 3 detects a face area of the patient from the video data as a skin-color area representing a body surface, and measures a heart rate based on changes in the color of the face area over time.

The calculation unit 4 calculates the reliability of the biometric information of the patient measured from the video data. For example, the calculation unit 4 can calculate the reliability of the measured biometric information based on movement information of the patient included in the video data of the patient.

In the case where the heart rate is measured from the face area of the video data of the patient as described above, if the patient moves his/her face, the angle between the light source and the face changes, so that the color of the face changes. If the color of the body surface changes due to the movement of the patient as described above, the reliability of the measured heart rate deteriorates.

The calculation unit 4 obtains the movement (movement information) of the face, for example, by tracking the coordinates of each part of the face of the patient, such as each of the eyes, the nose, and the outline of the face, and can determine that the reliability of the measured heart rate is low when the movement of the face is large and determine the reliability is high when the movement of the face is small.

The control unit 5 performs control so as to output, to the patient, instruction information for adjusting his/her state based on the calculated reliability. That is, the control unit 5 generates instruction information for guiding the patient in regard to his/her movement so that the doctor can obtain more reliable biometric information of the patient.

As described above, in the case where a heart rate is measured, if the patient moves his/her face, it affects the result of the measurement. Therefore, when the reliability calculated based on the movement information of the patient is low, the control unit 5 can generate, for example, an instruction “Do not move your face” so that the patient does not move his/her face.

Although the calculation unit 4 calculates the reliability of measured biometric information based on the movement information of the patient in the above-described example, the present disclosure is not limited to this example. For example, the calculation unit 4 can also calculate the reliability based on the size of the face area of the patient in the video data. For example, in the case of measuring a heart rate, since the measurement unit 3 measures the heart rate by using a face area of a patient included in video data, the video data needs to contain a face area having a predetermined size or larger.

When the face of the patient in the video data is smaller than the predetermined size, the calculation unit 4 can determine that the reliability is low. In this case, the control unit 5 can generate an instruction “Bring your face close to the camera” for the patient. By generating instruction information according to the reliability of measured biometric information as described above, it is possible to guide the patient in regard to his/her movement and thereby to obtain more reliable biometric information.

Further, when the room in which the patient is present is dark and hence changes in the color in the face area cannot be obtained, the calculation unit 4 can determine that the reliability is low. In this case, the control unit 5 can generate an instruction “Lighten the room” for the patient. Examples of instruction information for adjusting the state of a patient include, in addition to the above-described “instruction to bring the face to a standstill”, “instruction to bring the face closer”, and “instruction to adjust the brightness”, other various instructions for improving the reliability of biometric information and instructions necessary for the medical examination, such as “instruction to adjust the focus” and “instruction to show a part to be measured (e.g., direct a part to be measured toward the camera or the like)”.

The determination unit 6 acquires relevant information related to the patient included in the video data and determines biometric information to be measured based on one piece of relevant information or a combination of two pieces or more of relevant information. The relevant information may include medical record information, medical inquiry information, ability information indicating the ability to perform activities of daily living such as ADL (Activities of Daily Living), information indicating records of prescription, nursing care, rehabilitation and the like, and information on a department of a hospital or the like in which the patient usually undergoes a medical examination. The above-described information may be stored and managed in an external management server.

For example, when the determination unit 6 has acquired medical record information by accessing a management server, and found that it is stated that “breathing is hard” in the medical record information, it can determine “oxygen saturation” to be biometric information to be measured. Note that the determination unit 6 may determine a plurality of pieces of biometric information as items to be measured based on one piece of relevant information or a combination of two pieces or more of relevant information.

Further, the determination unit 6 may also determine that predetermined biometric information should not be used as an item to be measured based on one piece of relevant information or a combination of two pieces or more of relevant information. For example, types of biometric information to be measured are determined in advance for each department of a hospital or the like, and those that are not included in these types of biometric information determined in advance may be excluded from the items to be measured. For example, in the case of a patient in a dermatology department, the determination unit 6 can determine that any measurement for biometric information that is not necessary for the medical care in the dermatology department (e.g., measurement of oxygen saturation) should not be performed even when it is stated that “breathing is hard” in the medical record information.

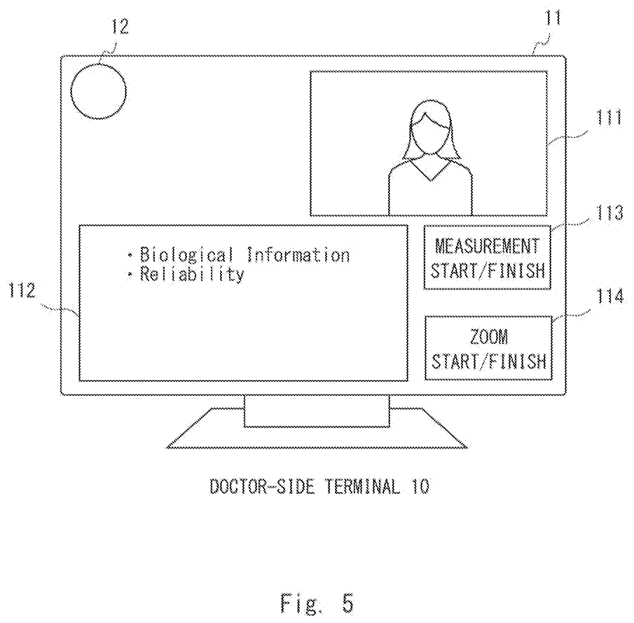

The display device 11 is an LCD (Liquid Crystal Display) or the like and displays various types of information. FIG. 5 is a diagram for explaining an example of contents displayed in the doctor-side terminal. As shown in FIG. 5, the display device 11 includes a video image display area 111, an information display area 112, a measurement button 113, and a zoom button 114.

In the video image display area 111, an image of a patient transmitted from the patient-side terminal 20 is displayed. In the information display area 112, biometric information measured from video data of the patient and its reliability are displayed. When the reliability is low, after instruction information for adjusting the state of the subject is output, the doctor can start the measurement of biometric information by the measurement unit 3 again by clicking the measurement button 113.

That is, in the example embodiment, the display device 11 serves as notification means for notifying the doctor, who is the user of the doctor-side terminal 10, of the reliability. Note that the notification means is not limited to any particular means as long as it can notify the doctor of the reliability through a voice, a notification sound pattern, a light emitting pattern, a vibration pattern, or the like.

Note that in the information display area 112, medical record information (e.g., a chronic disease, a medical history, examination information, and the like), inquiry information, and the like may be displayed. Further, the doctor-side terminal 10 may further include a voice analysis unit, and in the information display area 112, information related to contents of a conversation between the doctor and the patient, obtained through voice recognition may be displayed. For example, biometric information to be measured by the measurement unit 3 is determined from voice information “having a headache” spoken by the patient, and predetermined vital signs (a blood pressure, a heart rate, and the like) can be displayed.

Further, the information display area 112 may display candidate disease names according to the symptom by using the result of the voice recognition of the conversation between the doctor and the patient. Further, after the disease names are narrowed down according to the symptom, the information display area 112 may display recommendation information as to what kind of question should be asked in order to determine the disease name.

The shooting apparatus 12 is, for example, a video camera and can shoot (e.g., capture a video image of) the doctor who is using the doctor-side terminal 10. The shooting apparatus 12 shoots the doctor in a predetermined shooting range (shooting angle of view) at a predetermined shooting rate, and thereby generates moving image data composed of a plurality of time-series image data.

The storage device 13 is a non-volatile storage device, such as a hard disk drive (HDD), a solid-state drive (SSD), or a flash memory. The storage device 13 stores a program(s) for implementing the respective functions of the components provided in the information processing apparatus 1. When the information processing apparatus 1 executes this program, these functions of the components provided in the information processing apparatus 1 are implemented.

The storage device 13 may include a memory such as a RAM (Random Access Memory) and a ROM (Read Only Memory). When the information processing apparatus 1 executes the aforementioned program, the information processing apparatus 1 may execute the program after loading the program onto the memory, or may execute the program without loading it onto the memory. Further, the storage device 13 also has a function of storing information held by the components provided in the information processing apparatus 1, such as information necessary for the measurement unit 3 to measure biometric information and information necessary for the calculation unit 4 to calculate reliability.

The input device 14 is an apparatus that receives an operation input from the doctor, who is the user of the doctor-side terminal 10, such as a keyboard and mouse. The display device 11 and the input device 14 may be integrated and implemented as a touch panel. The doctor can, by using the input device 14, determine the start and end of the measurement of biometric information by clicking the measurement button 113.

As described above, in the example embodiment, medical care is carried out online between the doctor present in the hospital and the patient present in a place remotely located from the hospital, so that it is difficult for the patient, whose biometric information is to be measured, to know the timing of the start and end of the measurement that are determined by the doctor. Therefore, the control unit 5 preferably changes the instruction information for the patient according to the input (e.g., an input in regard to the start and end of the measurement of biometric information) to the input device 14.

For example, when the doctor inputs the start of the measurement of a heart rate, the control unit 5 can output information (e.g., a notification) “Measurement of a heart rate will start now. Please look squarely at the camera.” to the patient-side terminal 20. Further, when the doctor inputs the end of the measurement of biometric information, the control unit 5 can output information “Measurement has been finished” to the patient-side terminal 20. In this way, it is possible to enable the patient present in the remote place to know what the doctor will do, so that the patient can recognize at what timing he/she should not move and at what timing he/she can move. As described above, according to the example embodiment, it is possible to support smooth communication between the doctor and the patient in online medical care.

The communication apparatus 15 transmits and receives data to and from the external patient-side terminal 20 through, for example, a wireless transmission line. For example, the communication apparatus 15 transmits video data including the doctor (i.e., containing an image of the doctor) to the patient-side terminal 20 and receives video data including the patient (i.e., containing an image of the patient) from the patient-side terminal 20. Further, when information necessary for the above-described various calculations is stored in an external management server, the communication apparatus 15 receives the information from the management server.

The voice input device 16 is, for example, a microphone and collects a voice uttered (or spoken) by the doctor as voice data. Video data is generated by combining the moving image data generated by the shooting apparatus 12 and the voice data collected by the voice input device 16.

The information display area 112 can also display information or the like by using the result of the voice recognition of the voice data of the doctor collected by the voice input device 16 and voice information contained in the video data including the patient. The voice output device 17 is, for example, a speaker and reproduces (or plays back) the voice contained in the video data of the patient transmitted from the patient-side terminal 20.

The zoom instruction apparatus 18 outputs, to the patient-side terminal 20, a zoom instruction for controlling the zoom (i.e., the magnification factor or the like) of a shooting apparatus 22 that generates video data of the patient (which will be described later). The zoom means changing the angle of view to a telephoto mode or a wide-angle mode. For example, by changing the zoom state (focal length) of the optical system of the shooting apparatus 22, the diseased part of the patient can be enlarged.

When the doctor enlarges an abnormal part (diseased part) of the patient to be visually examined, the zoom instruction apparatus 18 can output a zoom instruction to the patient-side terminal 20 so as to enlarge the diseased part. The doctor can manually input the part to be enlarged to the zoom instruction apparatus 18 by clicking the zoom button 114 using the input device 14. Further, for example, when a heart rate is measured and it is desired to enlarge a specific face area, the face area may be automatically enlarged by using a face recognition technology. In this case, the zoom instruction apparatus 18 generates a zoom instruction so that the face area of the patient is extracted from the video data including the patient and the extracted face area is displayed in a predetermined size.

Further, the control unit 5 can change the instruction information to be provided to the patient according to the zoom state of the shooting apparatus 22. For example, when the doctor inputs an operation to zoom in the index finger of the right hand of the patient, which is the diseased part, the control unit 5 can output instruction information “I will examine your right index finger. Point the relevant part toward the camera and refrain from moving it as much as possible.” to the patient-side terminal 20 in order to guide the patient in regard to his/her movement. Further, when the doctor performs a zoom-out operation, the control unit 5 can output information “The examination is over. Thank you for your cooperation.” to the patient-side terminal 20 in order to cancel the instruction to guide the patient in regard to his/her movement.

Next, the patient-side terminal 20 will be described. As shown in FIG. 4, the patient-side terminal 20 includes an arithmetic apparatus 7, a display device 21, a shooting apparatus 22, a storage device 23, an input device 24, a communication apparatus 25, a voice input device 26, a voice output device 27, and a zoom control apparatus 28, all of which are connected to each other through a bus 29.

Note that since the general functions of the display device 21, the shooting apparatus 22, the storage device 23, the input device 24, the communication apparatus 25, the voice input device 26, and the voice output device 27 are the same as those of the display device 11, the shooting apparatus 12, the storage device 13, the input device 14, the communication apparatus 15, the voice input device 16, and the voice output device 17, respectively, descriptions thereof will be omitted.

The arithmetic apparatus 7 is, for example, an arithmetic processing apparatus such as a CPU and a GPU. The arithmetic apparatus 7 includes an instruction unit 8 as a logical functional block. The instruction unit 8 outputs instruction information for adjusting the state of the patient himself/herself from the doctor-side terminal 10. The instruction unit 8 can, for example, make the display device 21 (which will be described later) display the instruction information and make the voice output device 27 reproduce (or play back) the instruction information in the form of a voice.

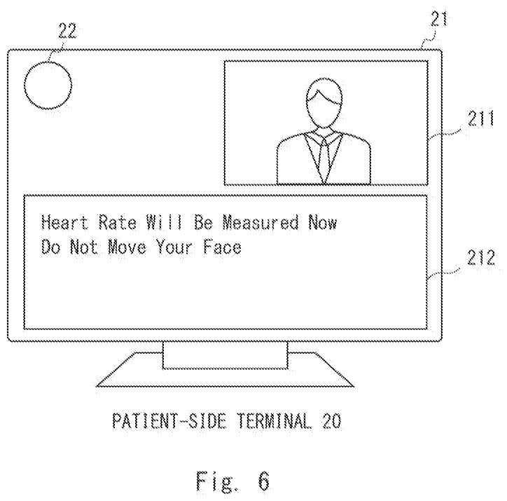

The display device 21 is an LCD or the like and displays various types of information. FIG. 6 is a diagram for explaining an example of contents displayed in the patient-side terminal. The display device 21 includes a video image display area 211 and an information display area 212. In the video image display area 211, video data including the doctor (i.e., containing an image of the doctor) transmitted from the doctor-side terminal 10 is displayed.

In the information display area 212, instruction information for the patient is displayed. As described above, when a heart rate is to be measured and the reliability calculated based on movement information of the patient is low, the control unit 5 generates an instruction “Do not move your face” so that the patient does not move his/her face. The instruction unit 8 receives this instruction “Do not move your face” and displays it in the information display area 212. By displaying the instruction information as described above, it is possible to guide the patient to adjust his/her own state and thereby to measure (i.e., obtain) reliable biometric information.

The instruction information for instructing the patient to adjust his/her state, which is displayed in the information display area 212, includes, in addition to the above-described instruction to bring the face to a standstill, an instruction to bring the face closer, an instruction to adjust the focus, an instruction to adjust the brightness, and the like which are issued according to the biometric information to be measured.

Further, for example, when it is determined, based on relevant information, to measure a heart rate based on the video image of the patient, the information display area 212 can display a notification or the like to notify the patient that a heart rate will be measured in advance. Further, when a zoom instruction is output from the zoom instruction apparatus 18, the information display area 212 can display a notification or the like to notify the patient of a part which will be zoomed in. The patient can know what is being measured from this notification and can refrain from moving his/her body part necessary for the measurement as much as possible.

As described above, the control unit 5 can change the instruction information provided to the patient according to the zoom state of the shooting apparatus 22. When the diseased part of the patient is to be zoomed in for a visual examination by the doctor, information as to where the diseased part of the patient to be zoomed in is displayed in the information display area 212. Further, when the visual examination by the doctor is finished and the diseased part of the patient is zoomed out, information indicating the end of the examination is displayed in the information display area 212.

Note that the output of instruction information is not limited to the example in which the instruction unit 8 outputs the instruction information by displaying it in the display device 21. For example, the instruction unit 8 may make the voice output device 27 output instruction information in the form of a voice. Further, the instruction unit 8 can also output instruction information in the form of a notification sound or light, or a vibration pattern.

The shooting apparatus 22 is, for example, a video camera and can shoot (e.g., capture a video image of) the patient who is using the patient-side terminal 20. The shooting apparatus 22 shoots the patient in a predetermined shooting range (shooting angle of view) at a predetermined shooting rate, and thereby generates moving image data composed of a plurality of time-series image data.

The storage device 23 is a non-volatile storage device, such as an HDD, an SSD, or a flash memory. The storage device 23 stores a program(s) for implementing the function of the instruction unit 8 provided in the arithmetic apparatus 7. When the arithmetic apparatus 7 executes this program, the function of the instruction unit 8 is implemented. Further, similarly to the storage device 13, the storage device 23 may include a memory such as a RAM and a ROM. The storage device 13 also has a function of storing data and the like necessary for the instruction unit 8 to perform the process for outputting instruction information.

The input device 24 is an apparatus that receives an operation input from the patient, who is the user of the patient-side terminal 20, such as a keyboard and mouse. The display device 21 and the input device 24 may be integrated and implemented as a touch panel.

The communication apparatus 25 transmits and receives data to and from the external doctor-side terminal 10 through, for example, a wireless transmission line. For example, video data including the doctor and video data including the patient are transmitted through the communication apparatus 25.

The voice input device 26 is, for example, a microphone and collects a voice uttered (or spoken) by the patient. Video data is generated by combining the moving-image data generated by the shooting apparatus 22 and the voice data collected by the voice input device 26. That is, this video data contains movement information and voice information of the patient. The voice output device 27 is, for example, a speaker and reproduces (or plays back) the voice contained in the video data of the doctor transmitted from the doctor-side terminal 10. Further, when instruction information for adjusting the state of the patient is output in the form of a voice, the voice output device 27 can reproduce (or play back) this instruction information in the form of a voice.

The zoom control apparatus 28 controls the zoom (i.e., the magnification factor or the like) of the shooting apparatus 22 in response to a zoom instruction from the doctor-side terminal 10. The zoom state of the shooting apparatus 22 is changed under the control of the zoom control apparatus 28, and a predetermined part of the patient is enlarged.

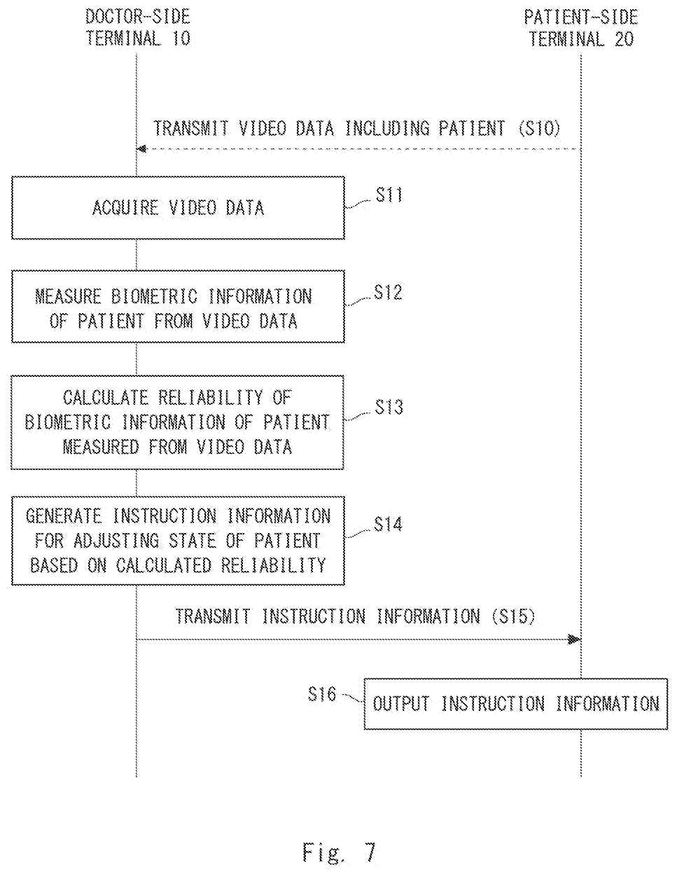

An information processing method according to an example embodiment will be described hereinafter with reference to FIG. 7. FIG. 7 is a sequence diagram for explaining an example of a flow of information processing according to an example embodiment. Firstly, the patient-side terminal 20 transmits video data including the patient to the doctor-side terminal 10 (S10). The acquisition unit 2 of the doctor-side terminal 10 acquires the video data including the patient (S11).

Then, the measurement unit 3 measures biometric information of the patient from the video data including the patient (S12). Note that in this process, the determination unit 6 may acquire relevant information related to the patient included in the video data and determine biometric information to be measured based on one piece of relevant information or a combination of two pieces or more of relevant information.

The calculation unit 4 calculates the reliability of the biometric information of the patient measured from the video data (S13). After that, the control unit 5 generates instruction information for adjusting the state of the patient based on the calculated reliability (S14) and transmits the generated instruction information to the patient-side terminal 20 (S15). The instruction unit 8 of the patient-side terminal 20 acquires this instruction information and output the acquired instruction information, for example, by displaying it in the display device 21 (S16).

As described above, according to the example embodiment, it is possible to provide, to the patient, the instruction information for adjusting the state of the patient according to the reliability of the measured biometric information. In this way, the patient is guided according to the instruction information, so that it is possible to improve the biometric information to be measured and thereby to improve the medical service.

Note that although the information processing system 100 includes the information processing apparatus 1 in the example shown in FIG. 3, the present disclosure is not limited to this configuration. For example, the information processing apparatus 1 can be implemented as a server installed in a data center or the like. Image data including a patient from the patient-side terminal 20 is transmitted to the server, and the server performs the above-described process for generating instruction information for adjusting the state of the patient.

Note that each of various functional blocks shown in the drawings, which perform various processes, can be implemented by hardware using a processor, a memory, and other circuits. Further, it is also possible to carry out the above-described processes by having a processor execute a program(s). Therefore, these functional blocks can be implemented in various ways by hardware alone, software alone, or a combination thereof, and are not limited to any of them. The aforementioned programs may be stored in any of various types of non-transitory computer readable media and supplied to the computer. The non-transitory computer readable media includes various types of tangible storage media. Examples of the non-transitory computer readable media include a magnetic recording medium (such as a flexible disk, a magnetic tape, and a hard disk drive), a magneto-optic recording medium (such as a magneto-optic disk), a CD-ROM (Compact Disc-ROM), a CD-R, a CD-R/W, and a semiconductor memory (such as a mask ROM (Read Only Memory), a PROM (Programmable ROM), an EPROM (Erasable PROM), a flash ROM, and a RAM (Random Access Memory)). Further, the programs may be supplied to computers by using various types of transitory computer readable media. Examples of the transitory computer readable media include an electrical signal, an optical signal, and an electromagnetic wave. The transitory computer readable media can be used to supply programs to a computer through a wired communication line such as an electric wire and an optical fiber or a wireless communication line.

Although the present disclosure is described above with reference to example embodiments, the present disclosure is not limited to the above-described example embodiments. Various modifications that can be understood by those skilled in the art can be made to the configuration and details of the present disclosure within the scope of the disclosure.

Further, the whole or part of the example embodiments disclosed above can be described as, but not limited to, the following supplementary notes.

### (Supplementary Note 1)

An information processing system comprising:
acquisition means for acquiring video data including a subject;
calculation means for calculating reliability of biometric information of the subject measured from the video data; and
instruction means for outputting, to the subject, instruction information for adjusting a state of the subject based on the calculated reliability.

### (Supplementary Note 2)

The information processing system described in Supplementary note 1, further comprising:
shooting means for shooting the subject and thereby generating the video data; and
zoom control means for controlling a zoom of the shooting means, wherein
the instruction means changes the instruction information according to a zoom state of the shooting means.

### (Supplementary Note 3)

The information processing system described in Supplementary note 1 or 2, further comprising input means for receiving an input of a start and/or an end of measurement of the biometric information, wherein
the instruction means changes the instruction information in response to an input to the input means.

### (Supplementary Note 4)

The information processing system described in any one of Supplementary notes 1 to 3, further comprising notification means for notifying a user of the reliability.

### (Supplementary Note 5)

The information processing system described in any one of Supplementary notes 1 to 4, further comprising determination means for acquiring relevant information related to the subject included in the video data and determining biometric information to be measured based on the relevant information.

### (Supplementary Note 6)

The information processing system described in any one of Supplementary notes 1 to 5, further comprising a first terminal and a second terminal, the first and second terminals being configured to perform two-way communication with each other, wherein
the first terminal comprises first display means for displaying a video image of the subject and the reliability, and
the second terminal comprises second display means for displaying the instruction information.

### (Supplementary Note 7)

An information processing apparatus comprising:
acquisition means for acquiring video data including a subject;
calculation means for calculating reliability of biometric information of the subject measured from the video data; and
control means for performing control so as to output, to the subject, instruction information for adjusting a state of the subject based on the calculated reliability.

### (Supplementary Note 8)

The information processing apparatus described in Supplementary note 7, wherein
the acquisition means acquires the video data generated by shooting the subject by shooting means connected through a network,
the information processing apparatus further comprises zoom instruction means for outputting a zoom instruction so as to control a zoom of the shooting means, and
the control means changes the instruction information according to a zoom state of the shooting means.

### (Supplementary Note 9)

The information processing system described in Supplementary note 7 or 8, further comprising input means for receiving an input of a start and/or an end of measurement of the biometric information, wherein
the instruction means changes the instruction information in response to an input to the input means.

### (Supplementary Note 10)

The information processing apparatus described in any one of Supplementary notes 7 to 9, further comprising notification means for notifying a user of the reliability.

### (Supplementary Note 11)

The information processing apparatus described in any one of Supplementary notes 7 to 10, further comprising determination means for acquiring relevant information related to the subject included in the video data and determining biometric information to be measured based on the relevant information.

### (Supplementary Note 12)

The information processing apparatus described in any one of Supplementary notes 7 to 11, wherein the control means controls a first terminal and a second terminal so that a video image of the subject and the reliability are displayed on a first display unit of the first terminal and the instruction information is displayed on a second display unit of the second terminal, the first and second terminals being configured to perform two-way communication with each other.

(Supplementary note 13)

An information processing method comprising:
acquiring video data including a subject;
calculating reliability of biometric information of the subject measured from the video data; and
outputting, to the subject, instruction information for adjusting a state of the subject based on the calculated reliability.

### (Supplementary Note 14)

The information processing method described in Supplementary note 13, further comprising:
controlling a zoom of shooting means for shooting the subject and thereby generating the video data; and
changing the instruction information according to a zoom state of the shooting means.

### (Supplementary Note 15)

The information processing method described in Supplementary note 13 or 14, further comprising changing the instruction information in response to an input to an input means for receiving an input of a start and/or an end of measurement of the biometric information.

### (Supplementary Note 16)

The information processing method described in any one of Supplementary notes 13 to 15, further comprising notifying a user of the reliability.

### (Supplementary Note 17)

The information processing method described in any one of Supplementary notes 13 to 16, further comprising acquiring relevant information related to the subject included in the video data and determining biometric information to be measured based on the relevant information.

### (Supplementary Note 18)

The information processing method described in any one of Supplementary notes 13 to 17, further comprising making a first display unit of a first terminal display a video image of the subject and the reliability, and making a second display unit of a second terminal display the instruction information, the first and second terminals being configured to perform two-way communication with each other.

### (Supplementary Note 19)

A non-transitory computer readable medium storing a program for causing a computer to perform:
a process for acquiring video data including a subject;
a process for calculating reliability of biometric information of the subject measured from the video data; and
a process for outputting, to the subject, instruction information for adjusting a state of the subject based on the calculated reliability.

### (Supplementary Note 20)

The non-transitory computer readable medium storing the program described in Supplementary note 19, wherein the program further causes the computer to perform:
a process for controlling a zoom of shooting means for shooting the subject and thereby generating the video data; and
a process for changing the instruction information according to a zoom state of the shooting means.

### (Supplementary Note 21)

The non-transitory computer readable medium storing the program described in Supplementary note 19 or 20, wherein the program further causes the computer to perform a process for changing the instruction information in response to an input to an input means for receiving an input of a start and/or an end of measurement of the biometric information.

### (Supplementary Note 22)

The non-transitory computer readable medium storing the program described in any one of Supplementary notes 19 to 21, wherein the program further causes the computer to perform a process for notifying a user of the reliability.

### (Supplementary Note 23)

The non-transitory computer readable medium storing the program described in any one of Supplementary notes 19 to 22, wherein the program further causes the computer to perform a process for acquiring relevant information related to the subject included in the video data and determining biometric information to be measured based on the relevant information.

### (Supplementary Note 24)

The non-transitory computer readable medium storing the program described in any one of Supplementary notes 19 to 23, wherein the program further causes the computer to perform a process for making a first display unit of a first terminal display a video image of the subject and the reliability, and making a second display unit of a second terminal display the instruction information, the first and second terminals being configured to perform two-way communication with each other.

### REFERENCE SIGNS LIST

1 INFORMATION PROCESSING APPARATUS
2 ACQUISITION UNIT
3 MEASUREMENT UNIT
4 CALCULATION UNIT
5 CONTROL UNIT
6 DETERMINATION UNIT
7 CALCULATION APPARATUS
8 INSTRUCTION UNIT
10 DOCTOR-SIDE TERMINAL
11 DISPLAY DEVICE
12 SHOOTING APPARATUS
13 STORAGE DEVICE
14 INPUT DEVICE
15 COMMUNICATION APPARATUS
16 VOICE INPUT DEVICE
17 VOICE OUTPUT DEVICE
18 ZOOM INSTRUCTION APPARATUS
19 BUS
20 PATIENT-SIDE TERMINAL
21 DISPLAY DEVICE
22 SHOOTING APPARATUS
23 STORAGE DEVICE
24 INPUT DEVICE
25 COMMUNICATION APPARATUS
26 VOICE INPUT DEVICE
27 VOICE OUTPUT DEVICE
28 ZOOM CONTROL APPARATUS
29 BUS
100 INFORMATION PROCESSING SYSTEM
101 ACQUISITION MEANS
102 CALCULATION MEANS
103 INSTRUCTION MEANS
111 VIDEO IMAGE DISPLAY AREA
112 INFORMATION DISPLAY AREA
113 MEASUREMENT BUTTON
114 ZOOM BUTTON
211 VIDEO IMAGE DISPLAY AREA
212 INFORMATION DISPLAY AREA
N NETWORK

What is claimed is:

1. An information processing system comprising:
at least one memory storing instructions, and
at least one processor configured to execute the instructions to;
acquire video data including a subject;
calculate reliability of biometric information of the subject measured from the video data; and
output, to the subject, instruction information for adjusting a state of the subject based on the calculated reliability.

2. The information processing system according to claim 1, wherein at least one processor is further configured to execute the instructions to:
shoot the subject and thereby generate the video data; and
control a zoom state of the shooting, wherein
the instruction information according to a zoom state of the shooting is changed.

3. The information processing system according to claim 1, wherein at least one processor is further configured to execute the instructions to receive an input of a start and/or an end of measurement of the biometric information, wherein
the instruction information is changed in response to the input.

4. The information processing system according to claim 1, wherein at least one processor is further configured to execute the instructions to notify a user of the reliability.

5. The information processing system according to claim 1, wherein at least one processor is further configured to execute the instructions to acquire relevant information related to the subject included in the video data and determine biometric information to be measured based on the relevant information.

6. The information processing system according to claim 1, further comprising a first terminal and a second terminal, the first and second terminals being configured to perform two-way communication with each other, wherein
the first terminal comprises first display configured to display a video image of the subject and the reliability, and
the second terminal comprises second display configured to display the instruction information.

7. An information processing apparatus comprising:
at least one memory storing instructions, and
at least one processor configured to execute the instructions to;
acquire video data including a subject;
calculate reliability of biometric information of the subject measured from the video data; and
perform control so as to output, to the subject, instruction information for adjusting a state of the subject based on the calculated reliability.

8. The information processing apparatus according to claim 7, wherein at least one processor is further configured to execute the instructions to;
acquire the video data generated by shooting the subject by shooting apparatus connected through a network,
output a zoom instruction so as to control a zoom of the shooting means apparatus, and
change the instruction information according to a zoom state of the shooting apparatus.

9. The information processing apparatus according to claim 7, wherein at least one processor is further configured to execute the instructions to;
receive an input of a start and/or an end of measurement of the biometric information, and
change the instruction information in response to an input.

10. The information processing apparatus according to claim 7, wherein at least one processor is further configured to execute the instructions to notify a user of the reliability.

11. The information processing apparatus according to claim 7, wherein at least one processor is further configured to execute the instructions to acquire relevant information related to the subject included in the video data and determine biometric information to be measured based on the relevant information.

12. The information processing apparatus according to claim 7, wherein at least one processor is further configured to execute the instructions to control a first terminal and a second terminal so that a video image of the subject and the reliability are displayed on a first display unit of the first terminal and the instruction information is displayed on a second display unit of the second terminal, the first and second terminals being configured to perform two-way communication with each other.

13.-18. (canceled)

19. A non-transitory computer readable medium storing a program for causing a computer to perform:
a process for acquiring video data including a subject;
a process for calculating reliability of biometric information of the subject measured from the video data; and
a process for outputting, to the subject, instruction information for adjusting a state of the subject based on the calculated reliability.

20. The non-transitory computer readable medium storing the program according to claim 19, wherein the program further causes the computer to perform:
a process for controlling a zoom of shooting apparatus for shooting the subject and thereby generating the video data; and
a process for changing the instruction information according to a zoom state of the shooting apparatus.

21. The non-transitory computer readable medium storing the program according to claim 19, wherein the program further causes the computer to perform a process for changing the instruction information in response to an input to an input device for receiving an input of a start and/or an end of measurement of the biometric information.

22. The non-transitory computer readable medium storing the program according to claim 19, wherein the program further causes the computer to perform a process for notifying a user of the reliability.

23. The non-transitory computer readable medium storing the program according to claim 19, wherein the program further causes the computer to perform a process for acquiring relevant information related to the subject included in the video data and determining biometric information to be measured based on the relevant information.

24. The non-transitory computer readable medium storing the program according to claim 19, wherein the program further causes the computer to perform a process for making a first display unit of a first terminal display a video image of the subject and the reliability, and making a second display unit of a second terminal display the instruction information, the first and second terminals being configured to perform two-way communication with each other.
